(19) United States
(12) Patent Application Publication
(10) Pub. No.: US 2026/0066324 A1
(43) Pub. Date: Mar. 5, 2026

(54) REDOX CYCLABLE MOLECULES FOR ENERGY STORAGE

(71) Applicant: MICROSOFT TECHNOLOGY LICENSING, LLC, Redmond, WA (US)

(72) Inventors: Jake Allen SMITH, Seattle, WA (US); Bichlien Hoang NGUYEN, Seattle, WA (US); Rhys Braginton Pettee OLSEN, Redmond, WA (US); Ernesto MARTINEZ BAEZ, Seattle, WA (US)

(21) Appl. No.: 18/822,160

(22) Filed: Aug. 31, 2024

(51) Int. Cl. H01M 8/18; C07D 241/12; C07D 335/02

(52) U.S. Cl. CPC H01M 8/188; C07D 241/12; C07D 335/02; H01M 2300/0025

(57) ABSTRACT

This disclosure provides redox cyclable molecules for energy storage. These molecules belong to either the 4H-pyran-4-ylidene family or include a six-membered aromatic ring with one nitrogen atom at position 1 (pyridinium family) or two nitrogen atoms at positions 1 and 4 (pyrazinium family) or at positions 1 and 3 (pyrimidinium family). Molecules in these families are used as analytes in redox flow batteries.

### BACKGROUND

A rechargeable battery is an electrochemical device that stores and releases electrical energy through reversible chemical reactions, allowing for multiple cycles of charging and discharging. Rechargeable batteries can be useful when the supply and demand for energy come at different times. One example application of rechargeable batteries is for temporary storage of energy generated by renewable energy sources when supply exceeds demand. Rechargeable batteries can also be useful to meet periodic peak demand that exceeds the capacity of the grid or other power source. One example application for rechargeable batteries is to provide energy smoothing for data centers or other large consumers of electricity.

One type of rechargeable battery is the redox flow battery (RFB). RFBs store energy in the physical separation of two charge-carrying species, an anolyte and catholyte. These batteries are characterized by their ability to decouple energy storage capacity from power output, allowing for flexible scaling. Due to their size and weight, RFBs are best suited for use in fixed locations, such as grid energy storage or industrial applications such as storing electricity from renewable energy sources or providing energy smoothing for a data center. The power and efficiency of an RFB is determined by its electrodes, conductivities of the electrolytes and kinetics of redox reactions. In current commercial implementations of RFBs, vanadium species are commonly used as the anolyte and catholyte. However, vanadium is expensive, there is limited market availability, and there are environmental concerns about the use of vanadium. Alternative chemistries for use in RFBs could address these and other problems. This disclosure is made with respect to these and other considerations.

### SUMMARY

This disclosure identifies two new classes of redox cyclable molecules that can function as charge carriers in RFBs. These redox cyclable molecules are small organic molecules. A first class of molecules is based on the 4H-pyran-4-ylidene family. The 4H-pyran-4-ylidene family refers to a class of chemical structures characterized by a six-membered ring characterized by an unsaturated, non-aromatic six-membered ring containing an oxygen, sulfur, or phosphorous heteroatom at the 1 position. A second class of molecules has a six-membered aromatic ring with one nitrogen atom at position 1 (pyridinium family) or two nitrogen atoms at positions 1 and 4 (pyrazinium family) or at positions 1 and 3 (pyrimidinium family). Members of the second class of molecules may be positively charged iminium ions or they may be present as non-ionic, solvent adduct species.

Both classes of molecules are suitable for use as anolytes in an RFB. The RFB may use organic or aqueous solutions. If aqueous, the first class of molecules and the second class of molecules have a redox potential within the useful range for aqueous RFB applications which is about −1.5 V to about +1.5 V versus a standard hydrogen electrode (SHE).

Multiple variations around the core structures are provided for both the first class of molecules and the second class of molecules. Some of the variations target improved aqueous solubility. Additionally, four specific molecules from the first class of molecules and three specific molecules from second class of molecules are provided.

RFBs containing either of these two classes of small organic molecules may be used in any application for which conventional RFBs are currently used. This includes storage of electricity generated by renewable energy sources and energy smoothing for data centers.

This Summary is provided to introduce a selection of concepts in a simplified form that are further described below in the Detailed Description. This Summary is not intended to identify key features or essential features of the claimed subject matter nor is it intended to be used to limit the scope of the claimed subject matter. The term “techniques,” for instance, may refer to system(s) and/or method(s) as permitted by the context described above and throughout the document.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items.

FIG. 1 shows a generalized structure of an RFB.

FIG. 2 shows structures of redox cyclable molecules with a structure based on 4H-pyran-4-ylidene.

FIG. 3 shows structures of redox cyclable molecules with structures based on pyrazinium.

FIG. 4 shows a general procedure for synthesizing (1,1-dioxido-4H-thiopyran-4-ylidene)malononitrile a member of the 4H-pyran-4-ylidene family.

FIG. 5 shows a general procedure for synthesizing 3-cyano-1-methylpyrazinium tetrafluoroborate a member of the pyrazinium family.

### DETAILED DESCRIPTION

RFB technology is a promising rechargeable battery design for flexible, long-term, and safe energy storage. Unlike static batteries, RFBs allow spatial separation of the reaction area (i.e., cell stack) and storage area (i.e., catholyte/anolyte tanks), this results in the power and capacity being independent of each other. The power is determined by the area of electrode in cell stack while the capacity is determined by the concentration of electrolytes and the volume of storage tanks.

A RFB generally includes two tanks, one containing the catholyte and one containing the anolyte, a reaction chamber with two electrodes where redox reactions of the redox species take place, an ion selective membrane, and pumps for moving the anolyte solution and catholyte solution. In principle, the concentration and redox potentials of catholyte and anolyte directly determine the capacity and voltage of the RFB, respectively. The stabilities of the catholyte, anolyte, electrodes, and membrane and the rates of electrolyte crossover determine the cycle life of the RFB. In addition, the power and coulombic efficiency of the RFB is determined by its electrodes, the conductivities of its electrolytes, and kinetics of their redox reactions. The membrane separates the redox species and facilitates the conduction of charge carriers, while allowing the flow of electrons through the external circuit. The membrane diminishes crossover between catholyte and anolyte and selectively allows the transport of charge-carrier ions.

RFBs that use vanadium species for both the catholyte and anolyte are the most common commercial implementation. However, there is on-going research to identify alternative charge carrying species. Alternative metal species such as iron have been used in RFBs. See Yu, Sicen, et al. “A Low-Cost Sulfate-Based All Iron Redox Flow Battery.” Journal of Power Sources, vol. 513, 2021, p. 230457. Metal-free designs that use polymers such as lignin have also been created. See Mukhopadhyay, Alolika et al. “Metal-Free Aqueous Flow Battery with Novel Ultrafiltered Lignin as Electrolyte.” ACS Sustainable Chemistry & Engineering, vol. 6(4), 2018, 5394-5400. Colloids have also been used as catholytes in RFBs. See Wei, Z., Huang, Z., Liang, G. et al. “Starch-mediated colloidal chemistry for highly reversible zinc-based polyiodide redox flow batteries.” Nat Commun vol. 15, 2024, p. 3841. Small organic molecules have also been used to create organic redox flow batteries (ORFBs). See Li, Zening, et al. “Recent Progress in Organic Species for Redox Flow Batteries.” Energy Storage Materials, vol. 50, 2022, pp. 105-138.

The redox cyclable molecules identified in this disclosure are less expensive to obtain than vanadium and have fewer environmental issues. Additionally, organic molecules of the pyrazinium and pyridinium families have the potential for higher energy density than vanadium species because they can store multiple electrons per molecule in organic solvent. This represents a significant improvement over the single electron transfer in vanadium systems. Furthermore, these molecules can be engineered to have tailored redox potentials and solubilities, enhancing the overall RFB performance.

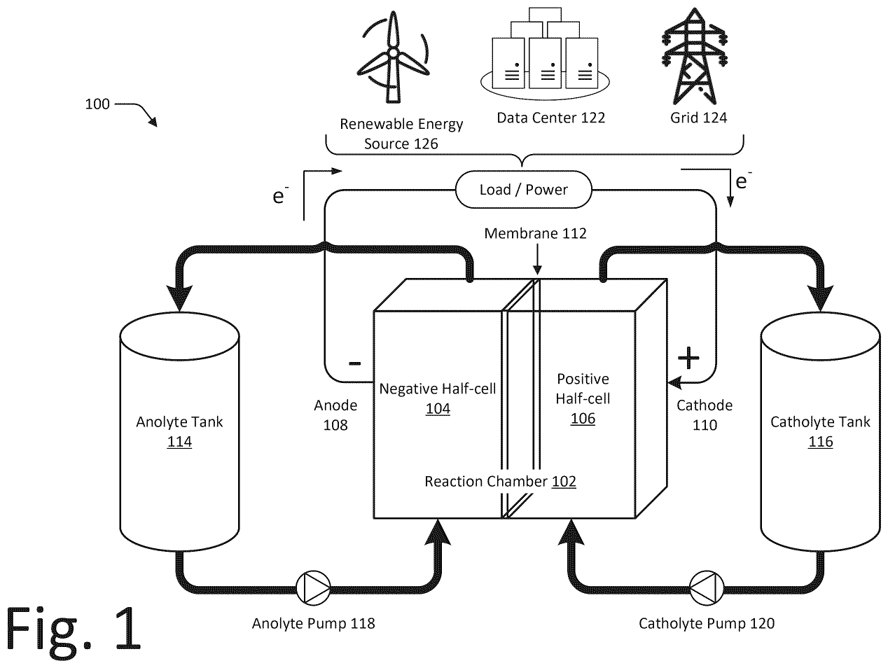

FIG. 1 shows a generalized structure of a RFB 100. The RFB 100 includes a reaction chamber 102 divided into a negative half-cell 104 and a positive half-cell 106. The negative half-cell 104 contains an anolyte solution and is in contact with an anode 108. The positive half-cell 106 contains catholyte solution and is in contact with the cathode 110. Typically, there is a membrane 112 separating the negative half-cell 104 and the positive half-cell 106. The membrane 112 maintains separation between the anolyte solution and the catholyte solution while allowing for transport of ions across the membrane 112. A catholyte is the electrolyte in a redox flow battery that undergoes reduction at the cathode during the battery's operation. An anolyte is the electrolyte that undergoes oxidation at the anode during the battery's operation. The transport of ions across the membrane 112 is essential for completing the electrical circuit needed to generate or store electricity.

The membrane 112 may be implemented as any one of a number of different types of membranes including, but not limited to, a proton exchange membrane, a cation exchange membrane, an anion exchange membrane, a porous membrane, or a bipolar membrane. A proton exchange membrane conducts protons between the positive and negative electrolytes. A cation exchange membrane allows cations to pass and blocks anions to maintain charge balance. One example of a cationic exchange membrane is Nafion®, a fluorinated sulfonic acid polymer of perfluorosulfonic acid that exhibits high cation conductivity, good chemical stability, low water permeability, and high selectivity. Another example of a cationic exchange membrane is Flemion®, a perfluorinated carboxylic acid polymer of perfluorocarboxylic acid that offers high cation conductivity, improved chemical stability, low water permeability, and high mechanical strength. An anion exchange membrane permits anions to move between electrolytes, facilitating ion exchange. A porous membrane contains pores for selective ion transport based on size, aiding in electrolyte separation. A bipolar membrane combines cation and anion exchange layers to enhance ion separation and battery efficiency.

In some implementations, the RFB 100 may be implemented without a membrane 112. A membrane-free RFB eliminates the need for a membrane to separate the electrolytes. Instead, it relies on careful design and control of fluid dynamics to keep the positive and negative electrolytes from mixing.

An anolyte tank 114 stores the anolyte solution and contributes to the capacity of the RFB 100. Similarly, a catholyte tank 116 stores the catholyte solution. Anolyte solution from the anolyte tank 114 is moved into the negative half-cell 104 by an anolyte pump 118. The catholyte solution is moved from the catholyte tank 116 to the positive half-cell 106 by a catholyte pump 120. There are return flow paths for both the anolyte solution and the catholyte solution. The anolyte solution and the catholyte solution may be aqueous solutions or non-aqueous solutions that use an organic solvent.

The anolyte solution comprises either the first class of molecules based on the 4H-pyran-4-ylidene family or the second class of molecules with a six-membered aromatic ring including one or two nitrogen atoms (pyridinium, pyrazinium, or pyrimidinium). Examples and characteristics of these classes of molecules are provided below.

The catholyte present in the catholyte solution may be any one of a number of different types of molecules or ions including, but not limited to, chloride, bromide, iodide, potassium ferrocyanide, ferrocene, 2,2,6,6-tetramethyl-1-piperidinyloxy (TEMPO), a benzoquinone, an anthraquinone, cerous methanesulfonate, or manganese dioxide. Iodide undergoes redox reactions, converting between iodide (I⁻) and iodine (I₂) as a catholyte. Potassium ferrocyanide participates in redox reactions by alternating between its oxidized form (ferricyanide, [Fe(CN)₆]³⁻) and its reduced form (ferrocyanide, [Fe(CN)₆]⁴⁻). In its oxidized form, ferrocene (Fc) is converted to ferrocenium (Fc⁺), and this redox couple facilitates electron transfer during an RBS's charge and discharge cycles. Ferrocene derivatives are particularly attractive for aqueous ORFBs due to their high solubility, stability, and tunable redox potentials. TEMPO is a stable free radical that can be oxidized to its cationic form (TEMPO⁺) and reduced back to its neutral form. This redox couple facilitates electron transfer during the charge and discharge cycles of the battery and is characterized by high solubility, stability, and well-defined redox potential. Benzoquinones, such as 1,4-benzoquinone, have high redox potential and good solubility in aqueous solutions. They alternate between their oxidized form (benzoquinone) and reduced form (hydroquinone), as a catholyte. Anthraquinones, such as anthraquinone-2,7-disulfonic acid (AQDS), have high chemical stability and tunable redox properties. They undergo redox reactions between their oxidized form (anthraquinone) and reduced form (anthrahydroquinone). Cerous methanesulfonate can function as a catholyte in redox flow batteries (RFBs) by participating in redox reactions where cerium ions alternate between different oxidation states. Specifically, cerium(III) ions (Ce³⁺) are oxidized to cerium(IV) ions (Ce⁴⁺) during the charging process and reduced back to Ce³⁺ during discharge. This redox couple is advantageous due to its high solubility in aqueous solutions and its stable redox potential, which contributes to efficient energy storage and release.

One or both of the anolyte solution and the catholyte solution may also contain a supporting electrolyte. Generally, but not always, the identity and concentration of the supporting electrolyte is identical in both the anolyte solution and the catholyte solution. Supporting electrolytes play an important role in RFBs by facilitating the flow of electrical charge between the positive and negative electrodes, enabling the efficient storage and release of energy. These electrolytes act as a conductive medium, allowing ions to move freely and complete the circuit, while also maintaining the chemical stability and overall performance of the battery. The choice of supporting electrolyte may depend on the type of solution used for the RFB 100, either aqueous or organic non-aqueous. Example supporting electrolytes that may be used with aqueous solutions include, but are not limited to, sodium hydroxide (NaOH), perchloric acid (HClO₄), potassium chloride (KCl), and sulfuric acid (H₂SO₄). Example supporting electrolytes that may be used with non-aqueous solutions include, but are not limited to, tetrabutylammonium tetrafluoroborate (TBATFB), tetraethylammonium phosphate (TEAP), lithium hexafluorophosphate (LiPF₆), and trifluoroacetic acid (TFA). The first class of molecules with a second class of molecules used as the anolyte are selected (e.g., structure including side groups) to have a redox potential within the electrochemical window of a solution of the supporting electrolyte.

The RFB 100 is capable of both generating current in response to a load or storing energy when charged by a power source. FIG. 1 illustrates electrons flowing from the anode 108 to the cathode 110 representing discharge of the battery. However, when charged by a power source, electrons will flow in the opposite direction from the cathode 110 to the anode 108 (not shown). In one implementation, the RFB 100 may be used to supply power to a data center 122. The data center 122, is but one example of a load that may be placed on the RFB 100. The RFB 100 may be used to supply power to any number of other types of facilities and machines.

When used as a power source for a data center 122, the RFB 100 may provide energy smoothing for the data center 122. Energy smoothing is a technique employed in data centers to mitigate fluctuations in electricity demand, ensuring a stable and consistent power supply. By reducing peak demand, improving power quality, and increasing efficiency, energy smoothing optimizes energy usage and minimizes strain on the electrical infrastructure. This is important in data centers, where electricity demand can vary significantly throughout the day.

To achieve energy smoothing, the data center 122 uses the RFB 100 to store and release electrical energy as needed, buffering against demand fluctuations and ensuring a reliable power supply. The RFB 100 receives energy from an external source such as the grid 124 or a renewable energy source 126. The grid 124 is a system comprising interconnected electrical networks designed for the generation, transmission, and distribution of electrical power to end users. By implementing energy smoothing techniques, data centers can reduce environmental impact, lower energy costs, and maintain optimal operating conditions. By storing excess energy during periods of low demand, the RFB 100 can effectively buffer against fluctuations in power usage. This is achieved through the reversible redox reactions of the anolyte and the catholyte, converting electrical energy into chemical energy. During periods of low demand, the RFB 100 stores excess energy converting electrical energy into chemical energy. When demand spikes, the process reverses, releasing stored energy to the data center 122 reducing peak demand placed on the grid 124 or other power source.

In an implementation, the RFB 100 can be used to store and release energy generated from renewable energy sources 126 such as solar panels or wind turbines. These types of renewable energy sources 126 are intermittent sources of power that have variations in power generation capacity. The RFB 100 can store large amounts of energy generated from renewable energy sources 126 during peak production times (e.g., sunny or windy periods) and release it when production is low (e.g., cloudy or calm periods). By buffering the energy output from renewable energy sources 126, the RFB 100 can help balance the grid by enabling a consistent power supply despite the variability of renewable energy generation.

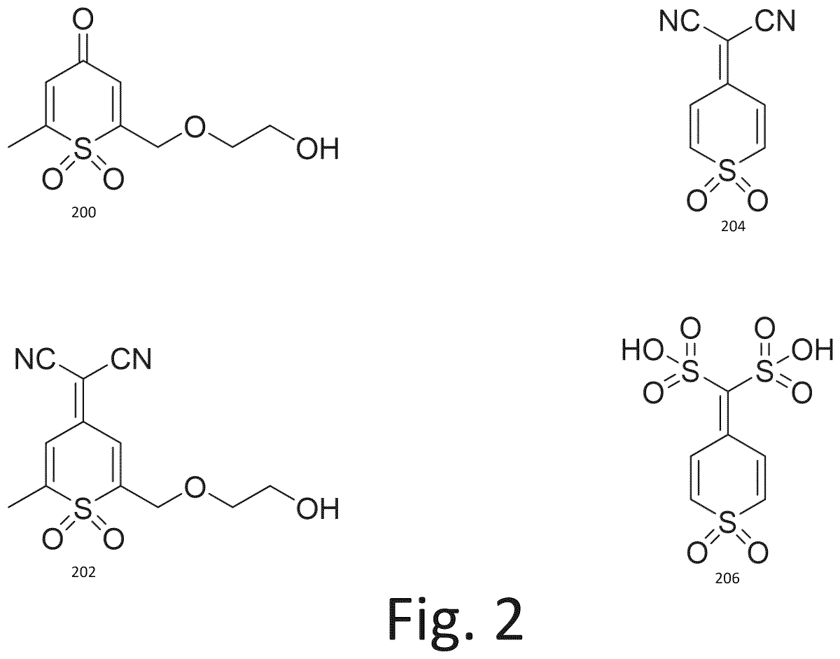

FIG. 2 shows four examples of the first class of molecules of the based on the 4H-pyran-4-ylidene family. Molecules of this class include the core structure:

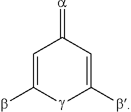

Under some conditions this core structure may be present as a stable reduced species such as a radical anion, 1-electron reduction product or a dianion, 2-electron reduction product:

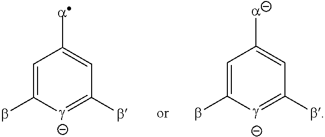

The α group may be any of O, S, or

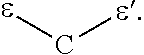

### If α is

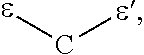

ε and ε′ may be the same or different
and are each independently one of H, CN, F,

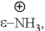

ε-CO₂H, ε-CO₂Me, ε-CO₂Bu, ε-CONH₂, ε-SO₃H, ε-PO₃H₂, methyl, ethyl, propyl, butyl, phenyl,

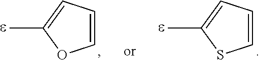

Although ε is shown in the representation of the groups, it is to be understood that it may also represent ε′. Also, ε, when shown, represents the point of attachment to the carbon in

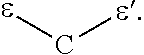

In one implementation, ε and ε′ are the same. In one particular implementation, α is oxygen. This is illustrated, for example, by compound 200. In one particular implementation, α is

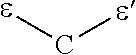

and ε and ε′ are both CN. This is illustrated, for example, by compounds 202 and 204. In one particular implementation, α is

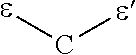

wherein ε and ε′ are both ε-SO₃H. This is illustrated, for example, by compound 206.

The β and β′ groups may be the same or different and are each independently one of H, methyl, ethyl, propyl, butyl,

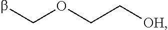

phenyl,

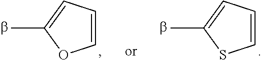

Although β is shown in the representation of the groups, it is to be understood that it may also represent β′. Also, β, when shown, represents the point of attachment to the aromatic six-membered ring. In one particular implementation, β and β′ are both H. This is illustrated, for example, by compounds 204 and 206. In one particular implementation, β is methyl and β′ is

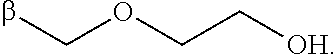

This is illustrated, for example, by compounds 200 and 202.

The γ group is one of O,

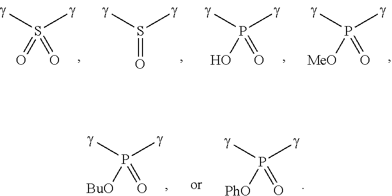

The notation γ-X-γ represents replacement of the γ in the core structure with the atoms of X. Thus,

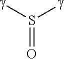

represents that the γ in the core structure is replaced by a sulfur that has a double bond to an oxygen. In one particular implementation, γ is

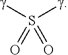

This is illustrated, for example, by compounds 200, 202, 204, and 206.

In one particular example, α is O, γ is

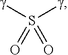

β is methyl and β′ is

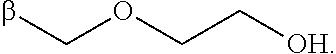

This is compound 200 shown in FIG. 2. In one particular example, α is

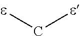

and ε and ε′ are both CN, γ is

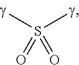

and β is methyl and β′ is

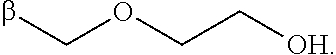

This is compound 202 shown in FIG. 2. In one particular example, α is

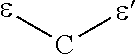

and ε and ε′ are both CN, γ is

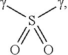

and β and β′ are both H. This is compound 204 shown in FIG. 2. In one particular example, α is

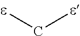

wherein ε and ε′ are both ε-SO₃H, γ is

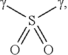

and ε and ε′ are both H. This is compound 206 shown in FIG. 2.

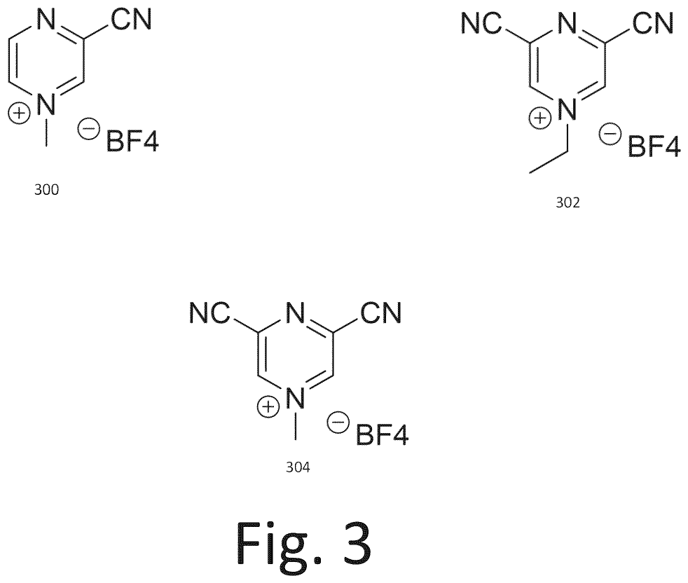

FIG. 3 shows three examples of the second class of molecules based on a six-membered aromatic ring with one nitrogen atom at position 1 (pyridinium family) or two nitrogen atoms at positions 1 and 4 (pyrazinium family) or at positions 1 and 3 (pyrimidinium family). Compounds of the second class that belong to the pyridinium family have the core structure:

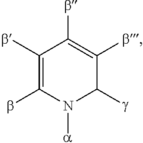

the cationic form

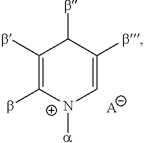

a the radical 1-electron product form

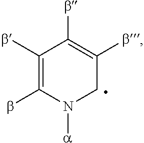

or the anionic 2-electron product form I.

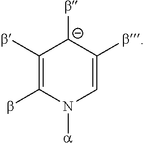

Compounds of the second class that belong to the pyrazinium family have the core structure:

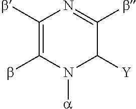

or the cationic form

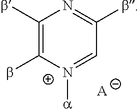

This is illustrated, for example, by compounds 300, 302, and 304.

Compounds of the second class that belong to the pyrimidinium family have the core structure:

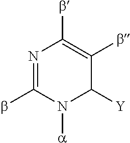

or the cationic form

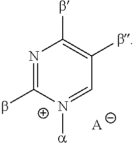

The areas of maximal electron density will change with the identities of the side groups. The cationic and solvent adduct forms may generally be understood to exist in equilibrium, and their relative concentrations will also change depending on operating conditions and the identities of the side groups. Thus, the cationic form and solvent adduct form are best understood not as separate structures, but as component species of the same core structure that may or may not be present depending on conditions and side groups.

In these representations, A⁻ is the counterion. The counterions are oppositely charged and stoichiometric to the anolyte species. The counterion, may be, but is not limited to, any of chloride (Cl⁻), bromide (Br⁻), iodide (I⁻), sulfate (SO₄²⁻), phosphate (PO₄³⁻), acetate (CH₃COO⁻), trifluoroacetate (CF₃COO⁻), tetrafluoroborate (BF₄⁻), or hexafluorophosphate (PF₆⁻). In one particular implementation, the compound of the second class is present as a cation and the counterion is tetrafluoroborate.

For all of the above core structures, the α group may be any of methyl, ethyl, propyl, butyl, or α-CH₂Ph (α representing the point of attachment to the nitrogen). In one particular implementation, the α group is methyl or ethyl.

The β, β′, β″, and (if present) β′″ groups are each independently one of H, CN, F,

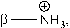

β-CO₂H, β-CO₂Me, β-CONH₂, methyl, ethyl, propyl, butyl, phenyl,

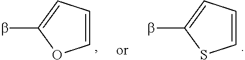

Here, β represents the point of attachment to the six-membered aromatic ring and may be any of β, β′, β″, or β′″. In one particular implementation, at least one of β, β′, β″, and β′″ is CN. In one particular implementation, at least one of β, β′, β″ and β′″ is H. In one particular implementation, at least one of β, β′, β″, and β′″ is CN and at least one of β, β′, β″, and β′″ is H. In one particular implementation, at least one of β, β′, β″, and β′″ is CN; at least one of β, β′, β″, and β′″ is H; and the others are either CN or H. Stated differently, all of β, β′, β″, and β′″ are either CN or H and at least one of β, β′, β″, and β′″ is CN and at least one other one of β, β′, β″, and β′″ is H.

The γ group is only present in the solvent adduct forms and is one of OH, OMe, or OBu. In ionic forms, the γ group detaches and is not present.

In one particular implementation, the core structure is

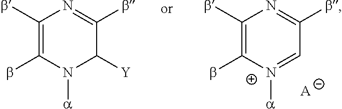

the α group is methyl, if present the γ group is OH, β and β′ are H, and β″ is CN. This is compound 300 shown in FIG. 3.

One particular implementation, the core structure is

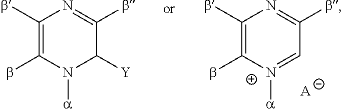

the α group is ethyl, if present the γ group is OH, β is H, and β′ and β″ are CN. This is compound 302 shown in FIG. 3.

In one particular implementation, the core structure is

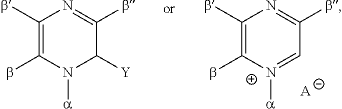

the α group is methyl, if present the γ group is OH, β is H, and β′ and β″ are CN. This is compound 304 shown in FIG. 3.

### EXAMPLES

Techniques described in the examples, and throughout this disclosure, are to be understood in the context of knowledge of one of ordinary skill in the art of organic chemical synthesis. Two synthetic pathways are described below, but a person of ordinary skill in the art will be able to modify the pathways described in these examples to produce any of the other compounds provided in this disclosure including all those illustrated in FIGS. 2 and 3. Examples of suitable techniques may be found in multiple reference materials including Carey, Francis A., and Richard J. Sundberg. Advanced Organic Chemistry: Part B: Reaction and Synthesis. 5th ed., Springer, 2007; Smith, Michael B., and Jerry March. March's Advanced Organic Chemistry: Reactions, Mechanisms, and Structure. 7th ed., Wiley, 2013; Kürti, László, and Barbara Czakó. Strategic Applications of Named Reactions in Organic Synthesis. 1st ed., Elsevier Academic Press, 2005; Larock, Richard C. Comprehensive Organic Transformations: A Guide to Functional Group Preparations. 2nd ed., Wiley-VCH, 1999; and Zweifel, George S., and Michael H. Nantz. Modern Organic Synthesis: An Introduction. 1st ed., W.H. Freeman, 2006.

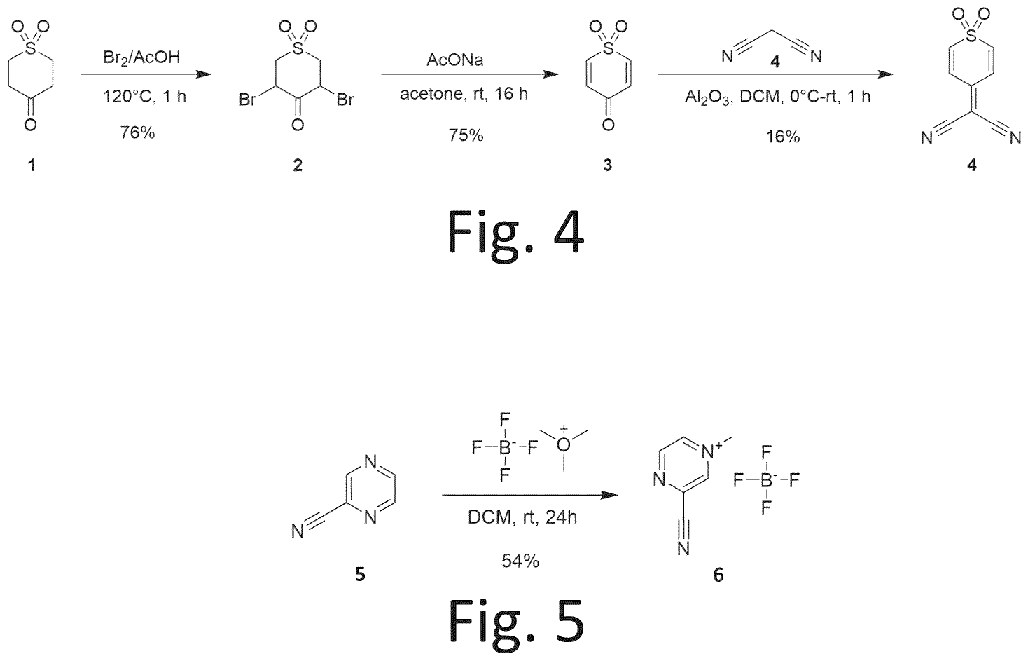

FIG. 4 shows a general procedure for synthesis of a one of the first class of molecules, a member of the 4H-pyran-4-ylidene, specifically (1,1-dioxido-4H-thiopyran-4-ylidene)malononitrile which is also shown as compound 204 in FIG. 2. The procedure begins by refluxing a solution of tetrahydro-4H-thiopyran-4-one 1,1-dioxide (compound 1, 10.0 g, 67.5 mmol, 1.0 eq) in AcOH (350 mL) and adding dropwise a solution of Br₂ (21.6 g, 135 mmol, 2.0 eq) in AcOH (100 mL). The reaction mixture is stirred at 120° C. for 1 hour. Upon cooling to room temperature, a white solid precipitates, and is collected by filtration, then washed with ether (15 mL×3) and dried to give compound 2 (15.6 g, 76%) as a white solid. This is illustrated below.

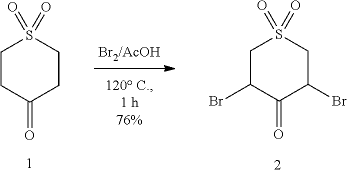 [SMILES: O=C1CCS(=O)(=O)CC1 ; O=C1C(Br)CS(=O)(=O)CC1Br]

The proton nuclear magnetic resonance spectrum for compound 2 is as follows: ¹H NMR (400 MHz, DMSO-d₆) δ 5.42 (dd, J=13.3, 5.3 Hz, 2H), 4.31-4.21 (m, 2H), 4.17-4.09 (m, 2H).

AcONa (12.9 g, 157.1 mmol, 8.1 eq) is added to a solution of 3,5-dibromotetrahydro-4H-thiopyran-4-one 1,1-dioxide (compound 2, 5.94 g, 19.4 mmol, 1.0 eq) in acetone (300 mL). The mixture is stirred at room temperature for 16 hours under nitrogen atmosphere. The reaction mixture is then diluted with H₂O (150 mL) and extracted with dichloromethane (DCM) (150 mL×3). The organic layers are washed with brine (100 mL×2), dried over anhydrous Na₂SO₄, filtered and concentrated under reduced pressure. The residue was triturated with EtOAc (10 mL) to give compound 3 (2.1 g, 75%) as white solid. This is illustrated below.

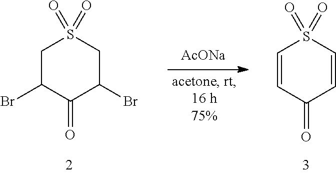 [SMILES: "AcONa" ; O=C1C(Br)CS(=O)(=O)CC1Br ; O=C1C=CS(=O)(=O)C=C1]

The proton nuclear magnetic resonance spectrum for compound 3 is as follows: ¹H NMR (400 MHz, DMSO-d₆) δ 8.00-7.94 (m, 2H), 6.73-6.67 (m, 2H).

To a solution of 4H-thiopyran-4-one 1,1-dioxide (compound 3, 500 mg, 3.47 mmol, 1.0 eq) and malononitrile (344 mg, 5.21 mmol, 1.5 eq) in DCM (50 mL) is added basic Al₂O₃ (5.0 g, 49 mmol, 14.1 eq) at 0° C. The reaction mixture is then stirred at room temperature for 1 hour. The reaction mixture is filtered through celite and the filter cake washed with DCM (15 mL×3). The combined filtrates are concentrated. The residue is recrystallized from EtOAc (3 mL) to give the final product, compound 4 (108.9 mg, 16%) as white solid. This is illustrated below.

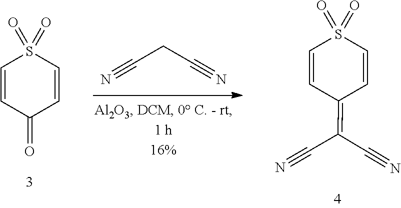 [SMILES: O=C1C=CS(=O)(=O)C=C1 ; N#CCC#N ; N#CC(C#N)=C1C=CS(=O)(=O)C=C1]

The proton nuclear magnetic resonance spectrum for compound 4 is as follows: ¹H NMR (400 MHz, CDCl₃-d) δ 7.32-7.27 (m, 2H), 7.06-7.01 (m, 2H).

FIG. 5 shows a general procedure for synthesis of a one of the second class of molecules, a member of the pyridinium family, specifically 3-cyano-1-methylpyrazinium. To a mixture of pyrazine-2-carbonitrile (compound 5, 500 mg, 4.76 mmol, 1.0 eq) in anhydrous DCM (20 mL) is added trimethyloxonium tetrafluoroborate (713 mg, 4.76 mmol, 1.0 eq). The mixture is stirred at room temperature for 24 hours. The mixture is then diluted with water (30 mL) and extracted with DCM (20 mL×3). The aqueous layer is lyophilized to give the final product, compound 6 (531 mg, 54%) as a brown solid.

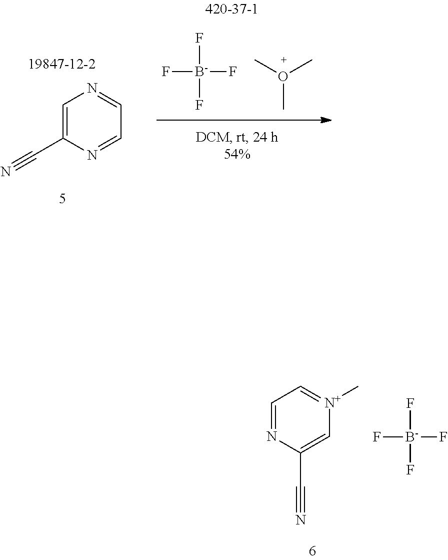

For compound 6, LC-MS: 120.0 [M+1]⁺ and proton nuclear magnetic resonance spectrum is ¹H NMR (400 MHz, CD₃OD-d4) δ 9.72 (s, 1H), 9.56 (s, 1H), 9.32 (d, J=2.8 Hz, 1H), 4.54 (s, 3H).

Calculated redox potentials for compounds shown in FIGS. 2 and 3 are provided in Table 1 below. The redox potentials were calculated using Density Functional Theory (DFT) and the Nernst equation. A rapid single-electron transfer relative to all proceeding mechanistic steps was assumed. Initial structures were relaxed with GFN2-xTB (GFN2-xTB is a semi-empirical quantum mechanical method for fast and accurate calculations of molecular structures, energies, and properties that uses the Generalized Functional for Nanostructures and second-generation tight-binding method with extended basis set. It is a simplified version of DFT, making it computationally efficient for large systems), then Self-Consistent Field (SCF) energies of the initial and +/− electron structures computed with the PW6B95 functional (Perdew-Wang exchange-correlation functional which includes a mix of 6% Hartree-Fock exchange and 95% Becke exchange) and def2tzvp basis set (including triple-zeta (TZ) quality for valence orbitals and additional polarization functions (p) for accurate description of molecular properties) with D3(BJ) dispersion (a dispersion correction method that includes a damping function that improves the accuracy of dispersion energies) and ddCOSMO (explicitly specifying the solvent as water) implicit solvent corrections.

The predicted redox potentials range from −4.71 V to +0.37 V. All values are provided as the standard electrode potential of a redox reaction, measured in volts relative to the Standard Hydrogen Electrode (SHE). For two compounds, one from the first class of compounds and one from the second class of compounds, experimentally generated redox potentials are identified from the literature. These predicted and experimental values are only slightly different. For (1,1-dioxido-4H-thiopyran-4-ylidene)malononitrile the difference is about +0.4 V. And for 3-cyano-1-methylpyrazinium the difference is even smaller, less than about +0.1 V. The experimental values were generated using an organic solvent and if an aqueous solvent was used (as assumed in the predictions) the difference would likely be even less.

These differences are interpreted in the context of the useful range of redox potentials for aqueous RFB applications which is about −1.5 V to about +1.5 V verses SHE. This range can be expanded if an organic solvent is used. Within this three-volt window a difference of less than 0.5 V is relatively small. Moreover, both the predicted and experimental values are well within the desired range of −1.5 V to +1.5 V verses SHE. Accordingly, the predicted redox potentials shown in Table 1 below are believed to be reasonably accurate predictions.

TABLE 1
Calculated and experimental redox potentials.

| | Predicted E^{o} | Experimental E^{o} |
|---|---|---|
| Compound | (V versus SHE) | (V versus SHE) |
| 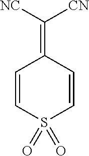 [SMILES: N#CC(C#N)=C1C=CS(=O)(=O)C=C1] | −0.36 | +0.038 (0.1M TBABF₄ in acetonitrile) (Detty et al.) |
| 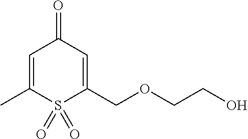 | −1.35 | |
| 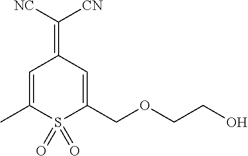 [SMILES: CC1=CC(=C(C#N)C#N)C=C(COCCO)S1(=O)=O] | −0.59 | |
| 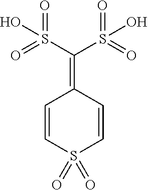 [SMILES: O=S1(=O)C=CC(=C(S(=O)(=O)O)S(=O)(=O)O)C=C1] | −4.71 | |
| 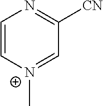 | −0.07 | +0.001 (0.1M TBABF₄ in acetonitrile) (S̆turala et al.) |
| 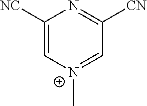 | +0.15 | |
| 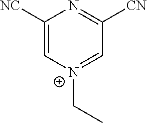 | +0.37 | |

Deity, Michael R., Raymond S. Eachus, et al. Electron Transport in 4H-1,1-Dioxo-4-(dicyanomethylidene)thioprans. Investigation of X-ray Structures of Neutral Molecules, Electrochemical Reduction to the Anion Radicals, and Absorption Properties and EPR Spectra of the Anion Radicals J. Org. Chem., vol. 60, no. 6, 1995, pp. 1674-1685.

Šturala, Jiří, Boháčová, S., Chudoba, J., Metelková, R., and Cibulka, R. Electron-Deficient Heteroarenium Salts: An Organocatalytic Tool for Activation of Hydrogen Peroxide in Oxidations. J. Org. Chem., vol. 80, no. 5, 2015, pp. 2676-2699 ILLUSTRATIVE EMBODIMENTS

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. An organic redox flow battery having an anolyte with the structure:

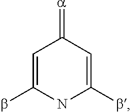

wherein, α is O, S, or

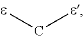

ε and ε′ are each independently H, CN, F,

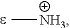

ε-CO₂H, ε-CO₂Me, ε-CO₂Bu, ε-CONH₂, ε-SO₃H, ε-PO₃H₂, methyl, ethyl, propyl, butyl, phenyl,

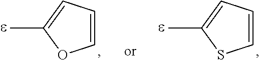

wherein, β and β′ are each independently H, methyl, ethyl, propyl, butyl,

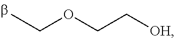

phenyl,

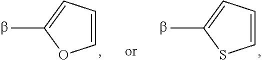

and
wherein, γ is O,

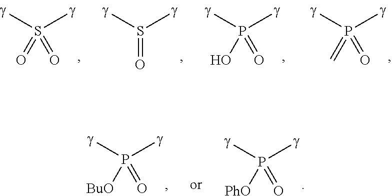

Clause 2. The organic redox flow battery of clause 1, wherein α is

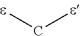

and ε and ε′ are the same.

Clause 3. The organic redox flow battery of clause 1 or clause 2, wherein α is O,

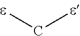

wherein ε and ε′ are both CN, or

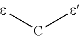

wherein ε and ε′ are both ε-SO₃H

Clause 4. The organic redox flow battery of any of clauses 1 to 3, wherein γ is

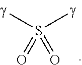

Clause 5. The organic redox flow battery of any of clauses 1 to 4, wherein β and β′ are both H or wherein β is methyl and β′ is

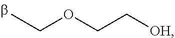

Clause 6. The organic redox flow battery of clause 1, wherein the anolyte has the structure

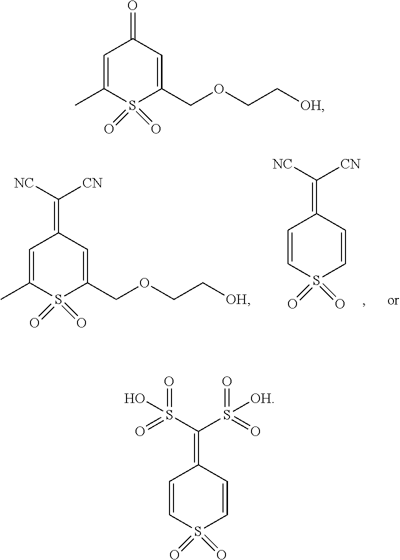 [SMILES: CC1=CC(=O)C=C(COCCO)S1(=O)=O ; CC1=CC(=C(C#N)C#N)C=C(COCCO)S1(=O)=O ; N#CC(C#N)=C1C=CS(=O)(=O)C=C1 ; O=S1(=O)C=CC(=C(S(=O)(=O)O)S(=O)(=O)O)C=C1]

Clause 7. The organic redox flow battery of any of clauses 1 to 6, further comprising a catholyte that is chloride, bromide, iodide, potassium ferrocyanide, ferrocene, 2,2,6,6-tetramethyl-1-piperidinyloxy (TEMPO), a benzoquinone, an anthraquinone, cerous methanesulfonate, or manganese dioxide.

Clause 8. The organic redox flow battery of any of clauses 1 to 7, further comprising a supporting electrolyte that is sodium hydroxide, perchloric acid, potassium chloride, sulfuric acid, tetrabutylammonium tetrafluoroborate, tetraethylammonium phosphate, lithium hexafluorophosphate, or trifluoroacetic acid.

Clause 9. The organic redox flow battery of any of clauses 1 to 8, wherein the anolyte has a redox potential of about −1.5 V to about +1.5 V versus a standard hydrogen electrode.

Clause 10. The organic redox flow battery of any of clauses 1 to 9, further comprising a membrane wherein the membrane is a proton exchange membrane, a cation exchange membrane, an anion exchange membrane, a porous membrane, or a bipolar membrane.

Clause 11. An organic redox flow battery having an anolyte with the structure:

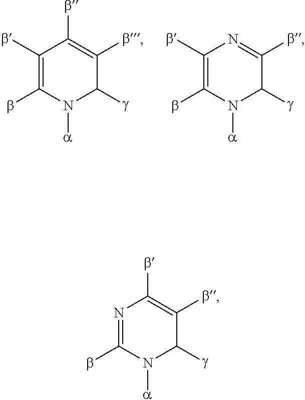

or a cation thereof
wherein α is methyl, ethyl, propyl, butyl, or α-CH₂Ph
wherein β, β′, β″, and β′″ are each independently H, CN, F,

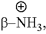

β-CO₂H, β-CO₂Me, β-CONH₂ methyl, ethyl, propyl, butyl, phenyl,

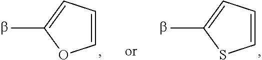

and
wherein γ is H, OH, OMe, or OBu.

Clause 12. The organic redox flow battery of clause 11, wherein the anolyte is a cation and the counter ion is chloride, bromide, iodide, sulfate, phosphate, acetate, trifluoroacetate, tetrafluoroborate, or hexafluorophosphate.

Clause 13. The organic redox flow battery of clause 11 or 12, wherein the anolyte comprises

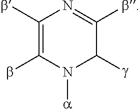

Clause 14. The organic redox flow battery of any of clauses 11 to 13, wherein α is methyl or ethyl.

Clause 15. The organic redox flow battery of any of clauses 11 to 14, wherein at least one of β, β′, β″, and β′″ is CN, at least one of β, β′, β″, and β′″ is H, and the others are either CN or H.

Clause 16. The organic redox flow battery of any of clauses 11 to 15, wherein γ is OH.

Clause 17. The organic redox flow battery of any of clauses 11 to 16, wherein the anolyte has the structure

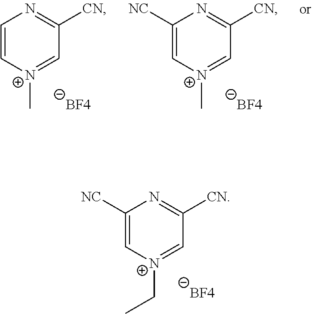

Clause 18. The organic redox flow battery of any of clauses 11 to 17, further comprising a catholyte that is chloride, bromide, iodide, potassium ferrocyanide, ferrocene, 2,2,6,6-tetramethyl-1-piperidinyloxy (TEMPO), a benzoquinone, an anthraquinone, cerous methanesulfonate, or manganese dioxide.

Clause 19. The organic redox flow battery of any of clauses 11 to 18, further comprising a supporting electrolyte that is sodium hydroxide, perchloric acid, potassium chloride, sulfuric acid, tetrabutylammonium tetrafluoroborate, tetraethylammonium phosphate, lithium hexafluorophosphate, or trifluoroacetic acid.

Clause 20. The organic redox flow battery of any of clauses 11 to 19, wherein the anolyte has a redox potential of about −1.5 V to about +1.5 V versus a standard hydrogen electrode.

### CONCLUSION

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced. As used herein, “approximately” or “about” or similar referents denote a range of ±10% of the stated value.

For ease of understanding, the processes discussed in this disclosure are delineated as separate operations represented as independent blocks. However, these separately delineated operations should not be construed as necessarily order dependent in their performance. The order in which the processes are described is not intended to be construed as a limitation, and unless other otherwise contradicted by context any number of the described process blocks may be combined in any order to implement the process or an alternate process. Moreover, it is also possible that one or more of the provided operations is modified or omitted.

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

Furthermore, references have been made to publications, patents, and/or patent applications throughout this specification. Each of the cited references is individually incorporated herein by reference for its particular cited teachings as well as for all that it discloses.

1. An organic redox flow battery having an anolyte with the structure: 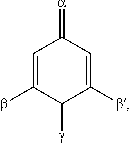
wherein, α is O, S, or 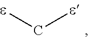
ε and ε are each independently H, CN, F, 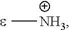
ε-CO₂H, ε-CO₂Me, ε-CO₂Bu, ε-CONH₂, ε-SO₃H₂, ε-PO₃H₂, methyl, ethyl, propyl, butyl, phenyl, 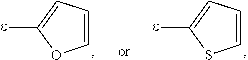
wherein, β and β′ are each independently H, methyl, ethyl, propyl, butyl, 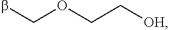
phenyl, 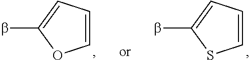
and
wherein, γ is O,

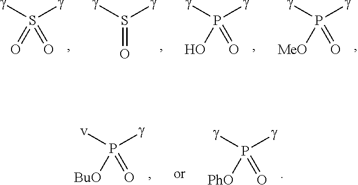

2. The organic redox flow battery of claim 1, wherein α is 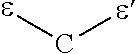
and ε and ε′ are the same.

3. The organic redox flow battery of claim 1, wherein α is O, 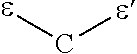
wherein ε and ε′ are both CN, or 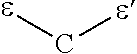
wherein ε and ε are both ε-SO₃H.

4. The organic redox flow battery of claim 1, wherein γ is

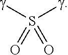

5. The organic redox flow battery of claim 1, wherein β and β′ are both H or wherein β is methyl and β′ is OH

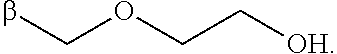

6. The organic redox flow battery of claim 1, wherein the anolyte has the structure

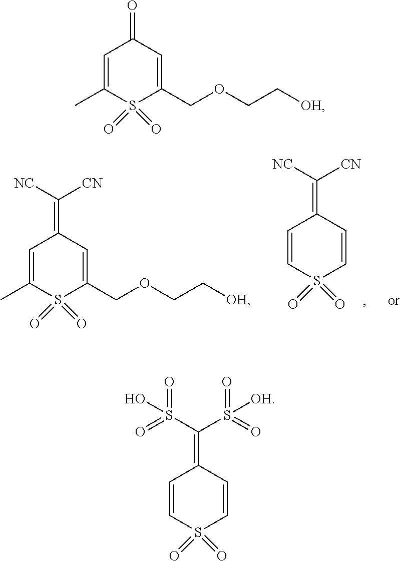 [SMILES: CC1=CC(=O)C=C(COCCO)S1(=O)=O ; CC1=CC(=C(C#N)C#N)C=C(COCCO)S1(=O)=O ; N#CC(C#N)=C1C=CS(=O)(=O)C=C1 ; O=S1(=O)C=CC(=C(S(=O)(=O)O)S(=O)(=O)O)C=C1]

7. The organic redox flow battery of claim 1, further comprising a catholyte that is chloride, bromide, iodide, potassium ferrocyanide, ferrocene, 2,2,6,6-tetramethyl-1-piperidinlyloxy (TEMPO), a benzoquinone, an anthraquinone, cerous methanesulfonate, or manganese dioxide.

8. The organic redox flow battery of claim 1, further comprising a supporting electrolyte that is sodium hydroxide, perchloric acid, potassium chloride, sulfuric acid, tetrabutylammonium tetrafluoroborate, tetraethylammonium phosphate, lithium hexafluorophosphate, or trifluoroacetic acid.

9. The organic redox flow battery of claim 1, wherein the anolyte has a redox potential of about −1.5 V to about +1.5 V versus a standard hydrogen electrode.

10. The organic redox flow battery of claim 1, further comprising a membrane wherein the membrane is a proton exchange membrane, a cation exchange membrane, an anion exchange membrane, a porous membrane, or a bipolar membrane.

11. An organic redox flow battery having an anolyte with the structure: 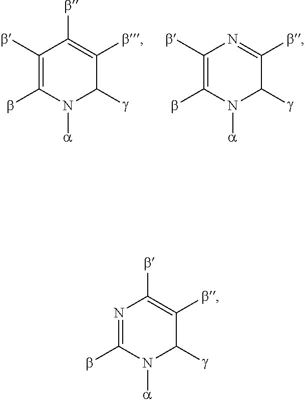
or a cation thereof
wherein α is methyl, ethyl, propyl, butyl, or α-CH₂Ph,
wherein β, β′, β″, and β′″ are each independently H, CN, F, 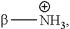
β-CO₂H, β-CO₂Me, β-CONH₂, methyl, ethyl, propyl, butyl, phenyl, 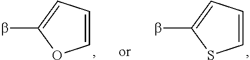
and
wherein γ is H, OH, OMe, or OBu.

12. The organic redox flow battery of claim 11, wherein the anolyte is a cation and the counter ion is chloride, bromide, iodide, sulfate, phosphate, acetate, trifluoroacetate, tetrafluoroborate, or hexafluorophosphate.

13. The organic redox flow battery of claim 11, wherein the anolyte comprises

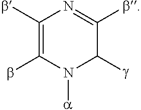

14. The organic redox flow battery of claim 11, wherein α is methyl or ethyl.

15. The organic redox flow battery of claim 11, wherein at least one of β, β′, β″, and β′″ is CN, at least one of β, β′, β″, and β′″ is H, and the others are either CN or H.

16. The organic redox flow battery of claim 11, wherein γ is OH.

17. The organic redox flow battery of claim 11, wherein the anolyte has the structure

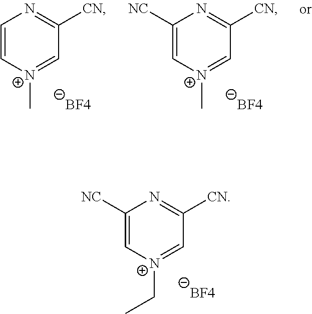

18. The organic redox flow battery of claim 11, further comprising a catholyte that is chloride, bromide, iodide, potassium ferrocyanide, ferrocene, 2,2,6,6-tetramethyl-1-piperidinyloxy (TEMPO), a benzoquinone, an anthraquinone, cerous methanesulfonate, or manganese dioxide.

19. The organic redox flow battery of claim 11, further comprising a supporting electrolyte that is sodium hydroxide, perchloric acid, potassium chloride, sulfuric acid, tetrabutylammonium tetrafluoroborate, tetraethylammonium phosphate, lithium hexafluorophosphate, or trifluoroacetic acid.

20. The organic redox flow battery of claim 11, wherein the anolyte has a redox potential of about −1.5 V to about +1.5 V versus a standard hydrogen electrode.
